(19) United States
(12) Patent Application Publication
(10) Pub. No.: US 2007/0126341 A1
(43) Pub. Date: Jun. 7, 2007

(54) El fiber and photocatalyst reaction vessel

(75) Inventor: Chihiro Kawai, Hyogo (JP)

Correspondence Address: VENABLE LLP, P.O. BOX 34385, WASHINGTON, DC, 20043-9998, US

(73) Assignee: Sumitomo Electric Industries, Ltd., Osaka (JP)

(21) Appl. No.: 10/580,204

(22) PCT Filed: Nov. 22, 2004
(86) PCT No.: PCT/JP04/17677

§ 371 Date: May 23, 2006

(51) Int. Cl. H01J 1/62; H01J 63/04

(52) U.S. Cl. 313/503

(57) ABSTRACT

A photocatalytic reaction is a reaction that occurs only on the surfaces of particles and, therefore, the particle surfaces must be uniformly irradiated with the ultraviolet light. However, a specific apparatus is required and a recovery cost is high in the exchange of photocatalyst particles. Furthermore, it is difficult to apply to a large reactor and, in particular, it is difficult to apply an external light source system. These problems are overcome. An EL fiber having a function of emitting ultraviolet light or visible light with a wavelength of 400 nm or less is characterized in that the cross-sectional structure of the fiber includes an internal electrode located at the center in a radius direction, an internal insulating layer disposed around the internal electrode, a light-emitting layer, an external electrode, and a protective layer disposed on an outermost surface, and the light is emitted by application of an alternating current electric field between the electrodes.

### TECHNICAL FIELD

The present invention relates to an EL fiber having a function of decomposing or sterilizing organic materials, bacteria, and the like and a photocatalytic reactor including the EL fiber.

### BACKGROUND ART

In consideration of recent environmental problems, photocatalytic materials which decompose or sterilize hazardous substances, bacteria viruses, and the like have been noted. A typical photocatalyst is TiO₂. In general, this is a material which performs a photocatalytic function by a ultraviolet light with a wavelength of 400 nm or less. Therefore, the photocatalyst can hardly exert a catalytic effect by sunlight having a small content of ultraviolet light.

Photocatalytic materials which function even by visible light with a wavelength exceeding 400 nm have also been developed. These are materials in which TiO₂ having an anatase type crystal system is doped with N, S, Mn, Fe, Co, Zn, Cu, or the like and, thereby, the absorption of the visible light is increased. Most of the materials come to perform the photocatalytic function even by the visible light. However, the performance is reduced to about 1/100th the performance of a combination of the ultraviolet light and anatase type TiO_{2.} Exceptionally, it has been reported that the performance is not significantly reduced for TiO₂ doped with a sulfur element. (Refer to 2003 Fall Meeting of The Electrochemical Society of Japan, Abstracts, The Electrochemical Society of Japan, page 322)

However, in either case, an external light source, e.g., a mercury lamp, must be used separately in order to allow these photocalysts to function. Therefore, reactors are hindered from being made compact and, in addition, a hazardous substance, mercury, must be used. Recently, a light-emitting diode (LED) that emits ultraviolet light is sometimes used instead of the mercury lamp.

On the other hand, a light-emitting fiber referred to as an EL fiber that emits light through electroluminescence has been known. FIG. 1 shows a conceptual structure thereof. Reference numeral 1 denotes an internal electrode, reference numeral 2 denotes an internal insulating layer, reference numeral 3 denotes a light-emitting layer, reference numeral 4 denotes an external insulating layer, reference numeral 5 denotes an external electrode, and reference numeral 6 denotes a protective layer. The external insulating layer 4 may not be disposed. High-energy level electrons referred to as hot electrons move in the light-emitting layer through the insulating layer by application of an alternating current voltage between the two electrodes, the hot electrons excite the semiconductor particles or specific ions included in the semiconductor particles in the light-emitting layer and, thereby, light emission occurs. Since the fiber emits light through electroluminescence, the fiber is referred to as an EL fiber. In general, commercially available EL fibers are simply green or blue visible-light emitting fibers, and are used for various illumination and the like. (Refer to Plastics, Rubber and Composites Progressing and Applications 1998. Vol. 27, No. 3, pages 160-165)

### DISCLOSURE OF INVENTION

A photocatalytic reaction is a reaction that occurs only on the surfaces of particles and, therefore, the particle surfaces must be uniformly irradiated with the ultraviolet light. However, there are problems as described below, for example.

(1) In the case where an object is a gas, TiO₂ particles serving as a photocatalyst must be floated in a reactor and, therefore, a specific apparatus is required. In the case of a liquid, the photocatalyst particles must be dispersed in the liquid. In this case, the cost of recovery is entailed in the exchange of photocatalyst particles.

(2) Since the ultraviolet light tends to be absorbed in air, a light source must be located nearby, and it is difficult to apply to a large reactor. Particularly, in the case where an object is a turbid liquid, the attenuation of the ultraviolet light is significant and, therefore, an external light source system cannot be applied.

It has been found that the above-described EL fiber has been improved on the basis of an original idea, so as to be allowed to have a function of decomposing or sterilizing organic materials, bacteria, and the like, and the above-described problems has been overcome. Consequently, the present invention has been made.

A first aspect of the present invention relates to an EL fiber primarily having a function of emitting ultraviolet light. The EL fiber has a function of emitting ultraviolet light or visible light with a wavelength of 400 nm or less, wherein the cross-sectional structure of the fiber includes an internal electrode located at the center in a radius direction, an internal insulating layer disposed around the internal electrode, a light-emitting layer, an external electrode, and a protective layer disposed on an outermost surface, and the light is emitted by application of an alternating current electric field between the electrodes. In the EL fiber, fluophor particles constituting the light-emitting layer are composed of a material that emits ultraviolet light. Bacteria and viruses may be directly decomposed or sterilized by the ultraviolet light. In particular, the ultraviolet light of 254 nm has been widely used for bactericidal lamps because DNAs of bacteria and viruses are directly destroyed. Therefore, the EL fiber that emits the ultraviolet light of 254 nm directly becomes an alternative to bactericidal lamps.

A second aspect of the present invention relates to an EL fiber having a function of emitting ultraviolet light or visible light. The EL fiber has a function of emitting ultraviolet light or visible light with a wavelength of 550 nm or less, wherein the cross-sectional structure of the fiber includes an internal electrode located at the center in a radius direction, an internal insulating layer disposed around the internal electrode, a light-emitting layer, an external electrode, a protective layer, and a particle layer or a thin film disposed on an outermost surface and having a photocatalytic function, and the light is emitted by application of an alternating current electric field between the electrodes. That is, the EL fiber and the photocatalyst are integrated.

In the second aspect, the emitted visible light or ultraviolet light is applied to the photocatalyst, and organic materials, bacteria, viruses, and the like are decomposed sterilized through the photocatalytic function. Therefore, the range of application is wider than that of the first aspect in which only the ultraviolet light is emitted.

Other aspects of the present invention relate to a photocatalytic reactor including the above-described EL fiber and, furthermore, a photocatalytic reactor having a structure in which the EL fiber and a photocatalytic fiber are combined in alternate position.

FIG. 1 shows an EL fiber to which an aspect of the present invention is applied. The internal electrode 1 may be an ordinary metal, and a cupper wire is used. The internal insulating layer 2 is used for uniformly applying an alternating current electric field to the light-emitting layer 3. Usually, a dielectric resin, e.g., Cyanoresin, is used alone, or a mixture of the dielectric resin and a ceramic powder, e.g., BaTiO₃, having a high dielectric constant is used. The thickness is a few tens of micrometers. For the external electrode 5, a transparent electrically conductive film formed from an indium-tin based oxide (ITO) or the like is used, or a NiCr alloy or the like having a thickness decreased to 0.1 μm or less is a candidate because the ultraviolet light or the visible light radiating from the light-emitting layer 3 must be transmitted.

The protective layer 6 is used for protecting the light-emitting layer 3 and the external electrode 5 from external environment factors, e.g., moisture, and the light radiating from the inside must also be transmitted. In the case where the light is visible light, an ordinary transparent resin may be used. However, for the ultraviolet light, a resin suitable for transmitting the ultraviolet light must be used. Examples thereof include ACRYLITE produced by MITSUBISHI RAYON CO., LTD. The protective layer 6 itself may be formed from a material having a photocatalytic function. For example, it is considered to apply a coating of dense TiO₂ by sputtering.

In the light-emitting layer 3 of a usual EL fiber, fluophor particles are dispersed in a dielectric resin, and the thickness of the light-emitting layer 3 is a few tens of micrometers. In the present aspect of the invention as well, the light-emitting layer 3 which radiates visible light with a wavelength exceeding 400 nm may be the same as that in the usual EL fiber. However, for the wavelength smaller than that, a resin may be deteriorated due to an extended period of use when the dielectric resin is used. Therefore, it is preferable that the dielectric ceramic is used instead of the resin. Various materials, e.g., BaTiO_{3,} SrTiO_{3,} and PbTiO₃, having a high dielectric constant are considered as the dielectric ceramic. That is, a layer having a type of core-shell structure results, in which fluophor particles are dispersed in the dielectric ceramic.

The fluophor is most important. ZnS based materials have been well known as fluophor materials which emit light with a high degree of efficiency through electroluminescence by being combined with the dielectric resin as in the aspects of the present invention, and have also been used as fluophors for general EL fibers. (Refer to Plastics, Rubber and Composites Progressing and Applications 1998. Vol. 27, No. 3, pages 160-165)

As shown in FIG. 2, ZnS is doped with Cl or Al as a second additional element. These additional elements form a donor level under a conduction band of ZnS. On the other hand, Cu, Ag, or the like is used as a first additional element for doping. These elements form an acceptor level on a valence band of ZnS. When ZnS is irradiated with energy, e.g., an electron beam or ultraviolet light, an electron in the valence band is once excited into the conduction band and, thereafter, is captured by the donor level. On the other hand, a hole newly generated in the valence band is captured by the acceptor level. The luminescence occurs by recombination of the electron located at the donor level and the hole located at the acceptor level. This is the luminescence of a type referred to as donor-acceptor (DA) luminescence, and is a luminescence mechanism capable of exhibiting an extremely high luminous efficacy. As indicated by Formula (1), basically, the luminescence wavelength is determined by the energy difference between the donor level and the acceptor level, and as this is increased, the wavelength of the luminescence is reduced. That is, the energy hv of luminescence is represented by
hv=Eg·(E_{D}+E_{A})·e²/(4πε₀εᵣr) (1)

where Eg represents band gap energy of ZnS, E_{D} represents binding energy of the donor, E_{A} represents binding energy of the acceptor, e represents an elementary charge, ε₀ represents a dielectric constant of vacuum, εᵣ represents relative electrostatic dielectric constant, and r represents a distance between the donor and the acceptor.

With respect to ZnS based fluophors having the above-described luminescence mechanism, ZnS:Ag,Cl and ZnS:Cu,Al have become commercially practical as a blue fluophor and a green fluophor, respectively. Therefore, it is possible to use these fluophors to allow the visible light with a wavelength of about 450 to 550 nm to be emitted.

As is clear from Formula (1), the luminescence wavelength is determined mainly on the basis of the band gap and the donor and acceptor levels of the semiconductor material. That is, in order to reduce the luminescence wavelength, it is necessary that (1) E_{g} is increased, (2) E_{D} is reduced, and (3) E_{A} is reduced. Among them, E_{D} is about 0.1 eV and is not significantly changed by elements used for doping. Furthermore, E_{A} is 0.7 eV when Ag is used for doping. Therefore, it is substantially most important to increase E_{g} in order to reduce the luminescence wavelength. Examples of additional elements for forming the acceptor level include Cu, Ag, Au, Li, Na, N, As, P, and Sb. Examples of additional elements for forming the donor level include Cl, Al, I, F, and Br.

It is believed that mainly two methods are used as the method for increasing the band gap energy (E_{g}). One method is to allow the matrix semiconductor to be a mixed crystal of the second component semiconductor having a band gap larger than that of ZnS (E_{g}=3.7 eV) and ZnS. Examples of the second component semiconductors include group II-IV compound semiconductors, while ZnS belongs to the same group. Selenides, e.g., MgSe (E_{g}=4.0 eV) and BeSe (E_{g}=4.7 eV), may also be used, but it is convenient to select sulfides similar to ZnS from the viewpoint of manufacturing ease. For example, E_{g} is 5.1 eV for MgS, 4.4 eV for CaS, and 4.3 eV for SrS, and these are preferable. In addition, BaS and BeS are also candidates. However, MgS is most preferable.

The other method for increasing the band gap is to reduce the size of the ZnS particle to a size on the order of nanometers. The quantum size effect is exerted due to reduction of the particle diameter and, thereby, the band gap is increased. As a matter of course, the particle diameter of the above-described mixed crystal may be reduced. In this case, the particle diameter may become larger than that in the case where a ZnS simple substance is used. The particle diameter suitable for exerting the quantum size effect is varied depending on E_{g} and E_{A}.

The luminescence wavelength of ZnS with 20 mole percent of MgS is 400 nm or less regardless of particle diameter. As described above, when the amount of MgS is increased, the limit of particle diameter tends to be removed. Conversely, the luminous efficacy may be reduced when the amount of MgS is increased. The same holds true for the other second component semiconductors. In that sense, the guideline for the particle diameter is 10 nm or less.

Examples of candidates for materials which emit ultraviolet light also include materials doped with Gd ions, e.g., Y₂O₃:Gd, Si—Y—O—N:Gd, and ZnF₂:Gd; GaN; and ZnO other than these ZnS based fluophors.

In the case where the ultraviolet light or visible light with a wavelength of 400 nm or less is used, the photocatalytic material may be anatase, rutile, or brookite type TiO_{2,} which are used usually. For the visible light with a wavelength exceeding 400 nm, TiO₂ doped with at least one element of N, S, Mn, Fe, Co, Zn, and Cu can be used as a visible-light sensitive photocatalyst. The material doped with S is most preferable, and the highest photocatalytic activity is exhibited.

The above-described products according to the present invention serve as compact light sources capable of directly radiating ultraviolet light or performing a photocatalytic function. Therefore, an efficient decomposition sterilization apparatus can be provided by disposing it in an object to be treated, e.g., a fluid in a confined portion that is not reached by external light or a liquid with high turbidity.

### BRIEF DESCRIPTION OF THE DRAWINGS

FIG. 1 is a conceptual diagram of an EL fiber to which the present invention can be applied.

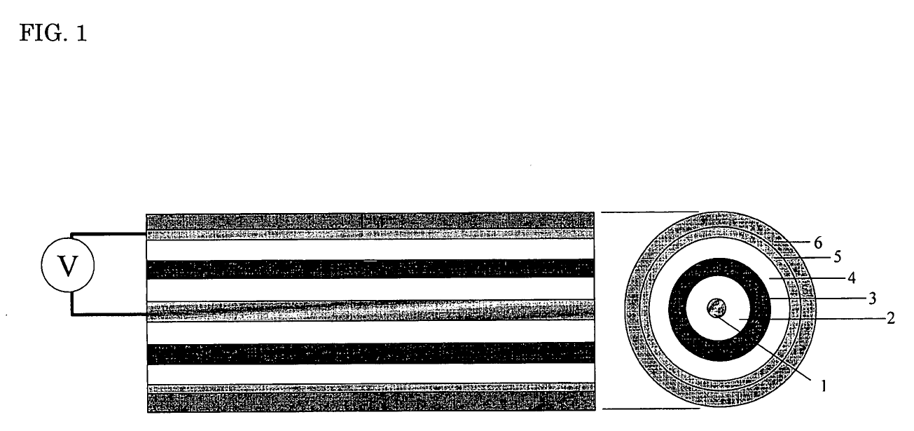

FIG. 2 is an explanatory diagram of a luminescence mechanism of a ZnS based fluophor.

FIG. 3 is an explanatory diagram of a photocatalytic reaction test.

FIG. 4 is an explanatory diagram of an example in which commercially available ultraviolet-emitting LEDs are arranged at intervals of 60 degrees.

FIG. 5 is an explanatory diagram of an example in which mercury lamps are arranged at intervals of 60 degrees.

FIGS. 6A and 6B are a plan view and a sectional view, respectively, of a woven fabric produced from EL fibers according to Example 1.

FIG. 7 is an explanatory diagram of Example 3.

FIG. 8 is an explanatory diagram of Comparative example relative to Example 3.

FIG. 9 is an explanatory diagram of another Comparative example relative to the above-described Example 3.

### BEST MODE FOR CARRYING OUT THE INVENTION

The present invention will be specifically described below with reference to Examples.

### EXAMPLE 1

A Cu wire having a diameter of 0.1 mm and a length of 1 m was used as a core electrode.

The following powders were prepared.

Insulating Layer Formation

BaTiO_{3:} average particle diameter 0.5 μm

Resin: produced by Shin-Etsu Chemical Co., Ltd., (trade name: Cyanoresin)

Fluophor

ZnS:Cu,Cl powder average particle diameter 0.5 μm (commercial item)

ZnS:Ag,Cl powder average particle diameter 0.5 μm (commercial item)

ZnS:Ag,Cl powder average particle diameter 3 to 15 nm

The powders were prepared by grinding a commercially available ZnS:Ag,Cl powder (average particle diameter 0.5 μm) in Ar at an acceleration of 150 G for various times through the use of a planetary ball mill apparatus (ball diameter was 50 μm).

Photocatalyst

Anatase type TiO₂ average particle diameter 0.05 μm (commercial item)

TiO₂:S average particle diameter 0.05 μm

A thiourea (CH₄N₂S) powder and Ti(OC₃H₇)₄ were mixed in ethanol, and vacuum concentration was conducted until a white slurry state resulted. Thereafter, firing was conducted at 600° C. for 2 hours in air, so that a powder was prepared. The amount of doping with S was specified to be 2 atomic percent relative to oxygen.

### (a) Formation of Insulating Layer

The resin was dispersed and dissolved into cyclohexanone so as to constitute 30 percent by volume. A BaTiO₃ powder was dispersed (30 percent by volume) in this solution. The resulting solution was applied to the Cu wire, the thickness was controlled at 30 μm with a revolving roller, and drying was conducted at 120° C. for 1 hour, so that an insulating layer was formed.

### (b) Formation of Light-emitting Layer

A solution was prepared, in which the resin was dispersed and dissolved into cyclohexanone so as to constitute 30 percent by volume. A fluophor powder was subjected to a dispersion treatment (30 percent by volume) in this solution in an Ar gas. The resulting solution was applied to the surface of the insulating layer of the item (a), the thickness was controlled at 40 μm with a revolving roller, and drying was conducted at 120° C. for 10 hours, so that a light-emitting layer was formed.

### (c) Formation of External Electrode

The resulting product was set in a sputtering apparatus, and the light-emitting layer surface was coated with an ITO electrode of 0.2 μm thickness at 130° C.

### (d) Formation of Protective Layer

A melt of ACRYLITE, which is an ultraviolet-transmitting resin, is applied, and the coating thickness was set at 100 μm with a revolving roller.

### (e) Formation of Photocatalytic Layer

A liquid was prepared, in which photocatalytic particles are dispersed in alcohol. An EL fiber was immersed therein, and was pulled out, so that the EL fiber surface was coated with TiO₂ particles.

### (f) Evaluation

(1) Luminous Efficacy

An alternating current electric field at 150 V and 400 Hz was applied between the core electrode and the ITO electrode of the EL fiber before being coated with the photocatalytic layer. The luminance of the luminescence was measured with a luminance meter or an ultraviolet illuminance meter, and the luminous efficacy was calculated from an electric power input.

(2) Photocatalytic Reaction Experiment

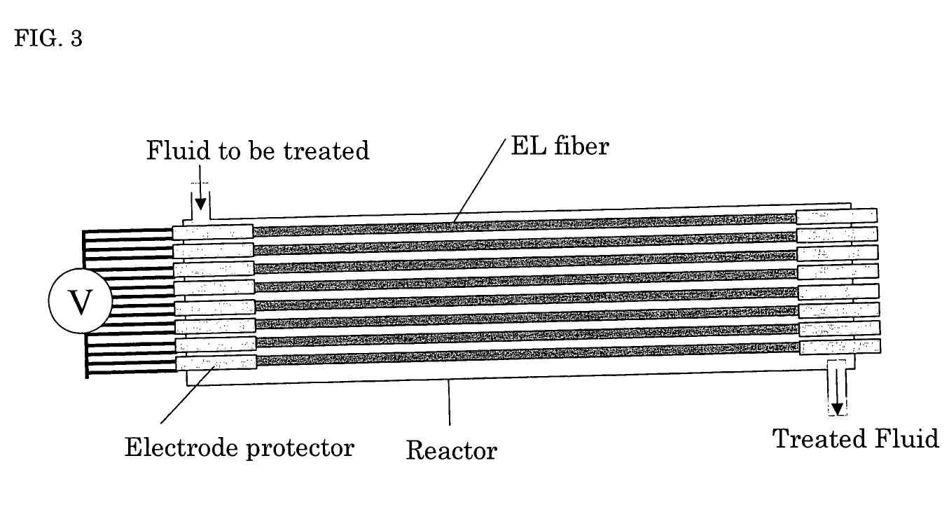

Five hundred TiO₂-coated EL fibers of 1 m length were tied in a bundle and set in a reactor of 50 cm diameter and 1 m length. As shown in FIG. 3, water containing trichloroethylene at a concentration of 100 ppm was introduced from an inlet of the reactor, and was circulated while being discharged from a separate outlet. The water was intentionally colored in advance by addition of 5% of India ink solution relative to the water, so that the liquid was allowed to have high turbidity. At this time, an alternating current electric field at 150 V and 400 Hz was applied between all the core electrodes and the ITO electrodes. The lapse of time until trichloroethylene was completely decomposed was measured.

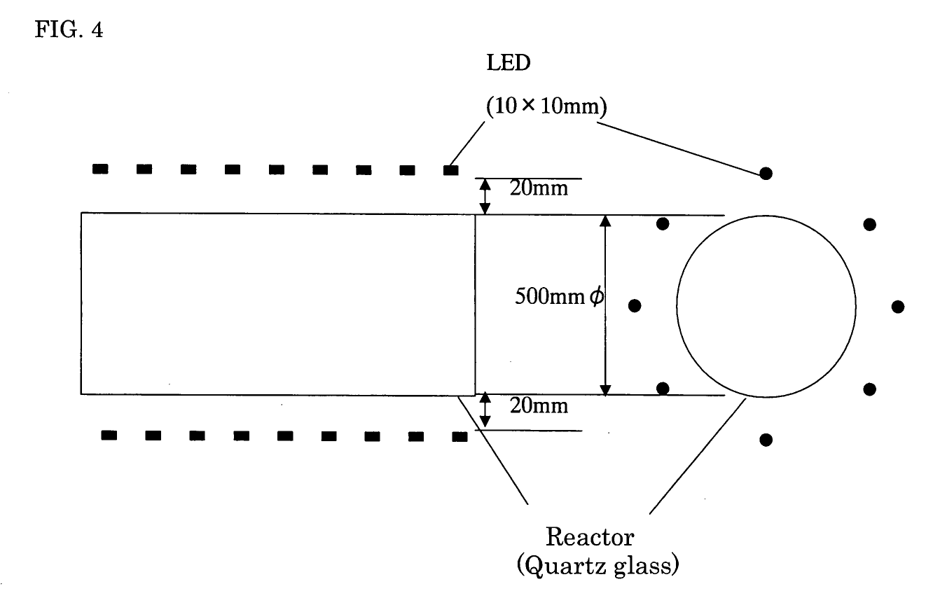

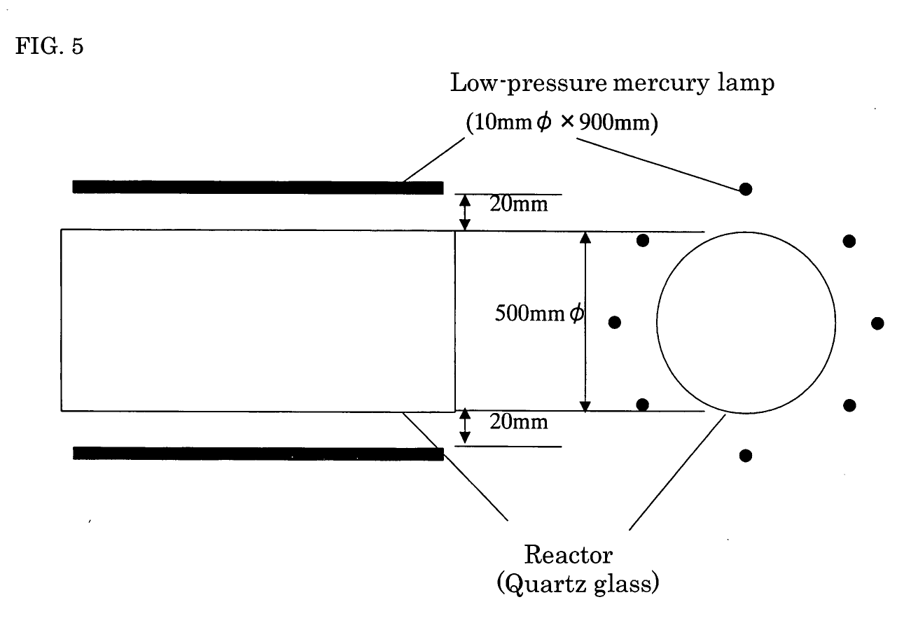

For the purpose of comparison, a device in which commercially available ultraviolet-emitting LEDs (luminescence wavelength 360 nm, output 50 mW) were arranged at intervals of 60 degrees (FIG. 4) and a device in which mercury lamps (luminescence wavelength 254 nm, output 100 mW) were arranged at intervals of 60 degrees and a pitch of 90 mm (FIG. 5) were prepared, and 100 g of the above-described anatase type TiO₂ particles were dispersed in the liquid contained in the reactor. The ultraviolet light was applied from the outside of the reactor, and the lapse of time until complete decomposition was measured.

The results are shown in Table 1.

TABLE 1

| | | Amount of | | | | | | | | | |
|---|---|---|---|---|---|---|---|---|---|---|---|
| | | second | | | | | | Lumines- | | | Lapse of |
| First | Second | semicon- | | | | | Fluophor | cence | | | time until |
| semicon- | semicon- | ductor | | | | | particle | wave- | Luminous | | complete |
| ductor | ductor | material | Donor | Acceptor | Grinding | Insulating | diameter | length | efficacy | Photo- | decomposi- |
| material | material | (mol %) | type | type | time (hr) | layer | (nm) | (nm) | (lm/W) | catalyst | tion (hr) |
| ZnS | None | 0 | Cl | Cu | Commer- | Cyanoresin + | 500 | 533 | 9.2 | TiO₂:S | 20.1 |
| | | | | | cial item | BaTiO₃ | | | | | |
| ZnS | None | 0 | Cl | Ag | Commer- | Cyanoresin + | 500 | 439 | 9.1 | TiO₂:S | 9 |
| | | | | | cial item | BaTiO₃ | | | | | |
| ZnS | None | 0 | Cl | Ag | 2 | Cyanoresin + | 20 | 437 | 9.7 | TiO₂:S | 8.9 |
| | | | | | | BaTiO₃ | | | | | |
| ZnS | None | 0 | Cl | Ag | 8 | Cyanoresin + | 5 | 414 | 9.8 | TiO₂:S | 8.6 |
| | | | | | | BaTiO₃ | | | | | |
| ZnS | None | 0 | Cl | Ag | 12 | Cyanoresin + | 3.5 | 391 | 10.5 | Anatase | 5 |
| | | | | | | BaTiO₃ | | | | | |
| ZnS | None | 0 | Cl | Ag | 12 | Cyanoresin + | 3 | 377 | 10.4 | Anatase | 1.1 |
| | | | | | | BaTiO₃ | | | | | |
| ZnS | None | 0 | Cl | Ag | 12 | Cyanoresin + | 3.5 | 391 | 10.5 | TiO₂:S | 5.3 |
| | | | | | | BaTiO₃ | | | | | |
| ZnS | None | 0 | Cl | Ag | 12 | Cyanoresin + | 3 | 377 | 10.4 | TiO₂:S | 1.2 |
| | | | | | | BaTiO₃ | | | | | |
| | GaN based | | | | | | | 360 | 40 | Anatase | 100< |
| | LED | | | | | | | | | | |
| | Low-pressure | | | | | | | 254 | 80 | Anatase | 100< |
| | mercury lamp | | | | | | | | | | |

For the external light source system, the experiment was conducted up to 100 hours at the maximum. However, trichloroethylene was not able to be completely decomposed. The reason for this is believed to be that the transparency of the liquid is low, the ultraviolet light does not adequately enter the inside of the reactor and, therefore, the photocatalyst does not perform its function.

On the other hand, when the product according to the present aspect of the invention was used, decomposition occurred. The decomposition can be effected by even visible light with a wavelength exceeding 400 nm when TiO₂:S is used as the photocatalyst. The decomposition capability was high when the ultraviolet light was used and, furthermore, the capability was increased as the wavelength of the ultraviolet light was reduced. The reason for this is believed to be that the photocatalyst can be adequately excited as the wavelength is reduced.

### EXAMPLE 2

A Cu wire having a diameter of 0.1 mm and a length of 1 m was used as a core electrode.

The following powders were prepared.

Insulating Layer Formation

Ba(OCH₃)₂

Ti(OC₂H₅)₄

Fluophor

ZnS—MgS:Ag,Cl powder average particle diameter 3 to 15 nm

The powders were prepared by mixing a commercially available ZnS:Ag,Cl powder (average particle diameter 0.5 μm) with a predetermined amount of MgS powder (average particle diameter 0.5 μm), and conducting grinding in Ar at an acceleration of 144 G for various times through the use of a planetary ball mill apparatus (ball diameter was 40 μm).

### (a) Formation of Insulating Layer

Alcohol solutions of Ba(OCH₃)₂ and Ti(OC₂H₅)₄ were individually vaporized and introduced into a CVD reactor. On the other hand, oxygen was introduced through another route. A reaction was conducted at a temperature of 900° C. and a pressure of 0.04 MPa for 2 hours, so that the Cu core electrode surface was coated with BaTiO₃ of 20 μm thickness.

### (b) Formation of Light-emitting Layer

In an Ar gas, a solution was prepared, in which the fluophor powder was dispersed in an alcohol solution (concentration 0.2 mol/l) mixed with equal moles of Ba(OCH₃)₂ and Ti(OC₂H₅)₄, the sample after the formation of the insulating layer was immersed in the resulting solution, and was pulled out. Firing was conducted in air at 900° C. for 30 minutes. This was repeated 30 times, so that a light-emitting layer of 20 μm thickness was formed, in which the fluophor particles were dispersed in BaTiO₃.

### (c) Formation of External Electrode

The resulting product was set in a sputtering apparatus, and the light-emitting layer surface was coated with an ITO electrode of 0.2 μm thickness at 530° C.

### (d) Formation of Protective Layer also Serving as Photocatalyst

Each of anatase type TiO₂ of 5 μm thickness and TiO₂:S, in which TiO₂ was doped with S by 2 atomic percent relative to oxygen, of 5 μm thickness was formed at 600° C. by a sputtering method so as to serve as a protective layer.

### (f) Evaluation

(1) Luminous Efficacy

An alternating current electric field at 200 V and 300 Hz was applied between the core electrode and the ITO electrode of the EL fiber before being coated with the photocatalytic layer. The luminance of the luminescence was measured with a luminance meter or an ultraviolet illuminance meter, and the luminous efficacy was calculated from an input electric power.

(2) Photocatalytic Reaction Experiment

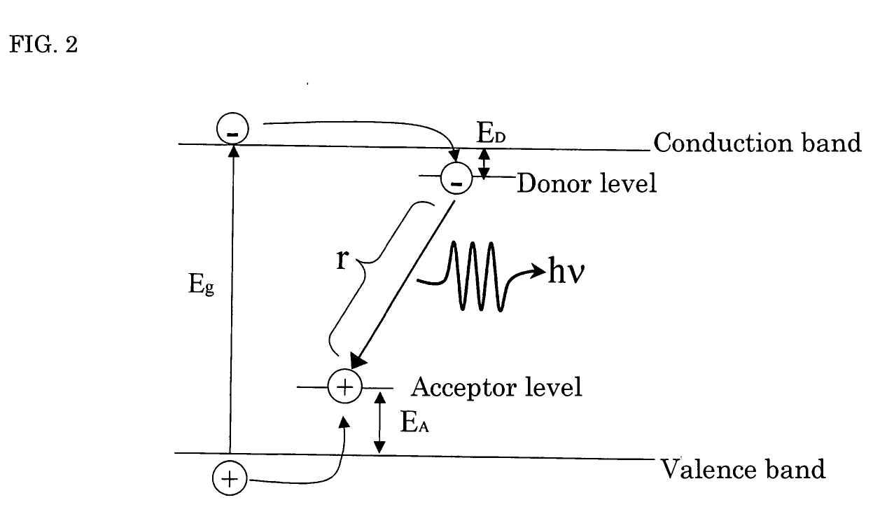

Five hundred EL fibers of 1 m length were tied in a bundle and disposed in a reactor of 50 cm diameter and 1 m length. As shown in FIG. 2, water containing acetaldehyde at a concentration of 180 ppm was introduced from an inlet of the reactor, and was circulated while being discharged from a separate outlet. The water was intentionally colored in advance by addition of 10% of India ink solution relative to the water, so that the liquid was allowed to have high turbidity. At this time, an alternating current electric field at 200 V and 300 Hz was applied between all the core electrodes and the ITO electrodes. The lapse of time until acetaldehyde was completely decomposed was measured.

The results are shown in Table 2.

TABLE 2

| | | Amount | | | | | | | | | |
|---|---|---|---|---|---|---|---|---|---|---|---|
| | | of second | | | | | | Lumines- | | | Lapse of |
| First | Second | semicon- | | | | | Fluophor | cence | | | time until |
| semicon- | semicon- | ductor | | | | | particle | wave- | Luminous | | complete |
| ductor | ductor | material | Donor | Acceptor | Grinding | Insulating | diameter | length | efficacy | Photo- | decomposi- |
| material | material | (mol %) | type | type | time (hr) | layer | (nm) | (nm) | (lm/W) | catalyst | tion (hr) |
| ZnS | MgS | 20 | Cl | Ag | 4 | BaTiO₃ | 8.7 | 399 | 10.9 | Anatase | 13.9 |
| ZnS | MgS | 40 | Cl | Ag | 4 | BaTiO₃ | 8.7 | 373 | 11.6 | Anatase | 2.2 |
| ZnS | MgS | 50 | Cl | Ag | 4 | BaTiO₃ | 8.7 | 360 | 12 | Anatase | 0.6 |
| ZnS | MgS | 20 | Cl | Ag | 4 | BaTiO₃ | 8.7 | 399 | 10.9 | TiO₂:S | 6.8 |
| ZnS | MgS | 40 | Cl | Ag | 4 | BaTiO₃ | 8.7 | 373 | 11.6 | TiO₂:S | 1 |
| ZnS | MgS | 50 | Cl | Ag | 4 | BaTiO₃ | 8.7 | 360 | 12 | TiO₂:S | 0.88 |

Since the fluophor was made to be a ZnS—MgS mixed crystal system, the luminescence wavelength was further reduced, and the decomposition rate was increased. Since the dielectrics in the insulating layer and the light-emitting layer were made to be BaTiO₃ having a high dielectric constant, high luminous efficacy was achieved.

The photocatalytic function was able to be performed even when TiO₂ was used as the protective layer.

### EXAMPLE 3

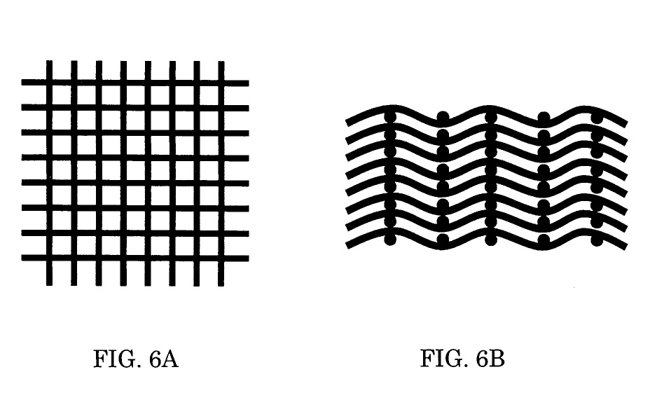

The EL fiber of Example 1 was subjected to two-dimensional satin weave at a pitch of 3 mm, so that a woven fabric having a size of 500 mm×500 mm was prepared. FIG. 6A is a plan view and FIG. 6B is a sectional view.

A woven fabric (produced by UBE INDUSTRIES, LTD.) formed from a photocatalytic fiber and cut to 500 mm×500 mm was prepared.

The EL fiber woven fabric and the photocatalytic woven fabric were stacked in alternate position, and 50 layers each thereof were laminated, so that a photocatalytic device was prepared.

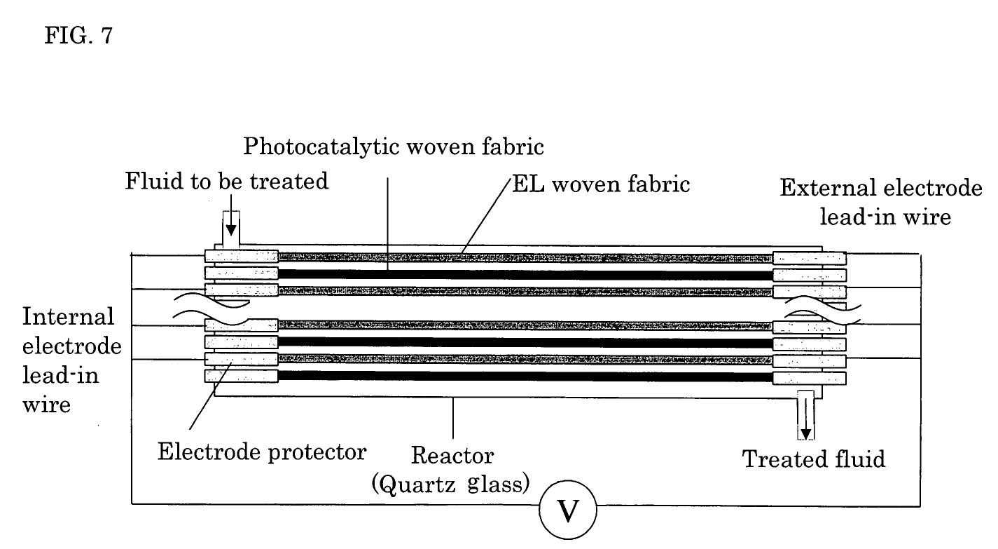

The device was set in a container (a reactor having a thickness of 70 mm and a cross-sectional area of 500 mm×500 mm) shown in FIG. 7. One type of dioxins, 2,3′,4,4′,5—Pc—CB, was dissolved into water and, thereby, 30 l of solution having a concentration of 100 pg/l was prepared. At this time, the water was intentionally colored in advance by addition of 10% of India ink solution relative to the water, so that the liquid having high turbidity was prepared.

An alternating current electric field at 200 V and 500 Hz was applied between the electrodes while the resulting liquid was circulated at a flow rate of 2.5 l/min. The lapse of time until dioxin was completely decomposed was measured up to 100 hours at the maximum.

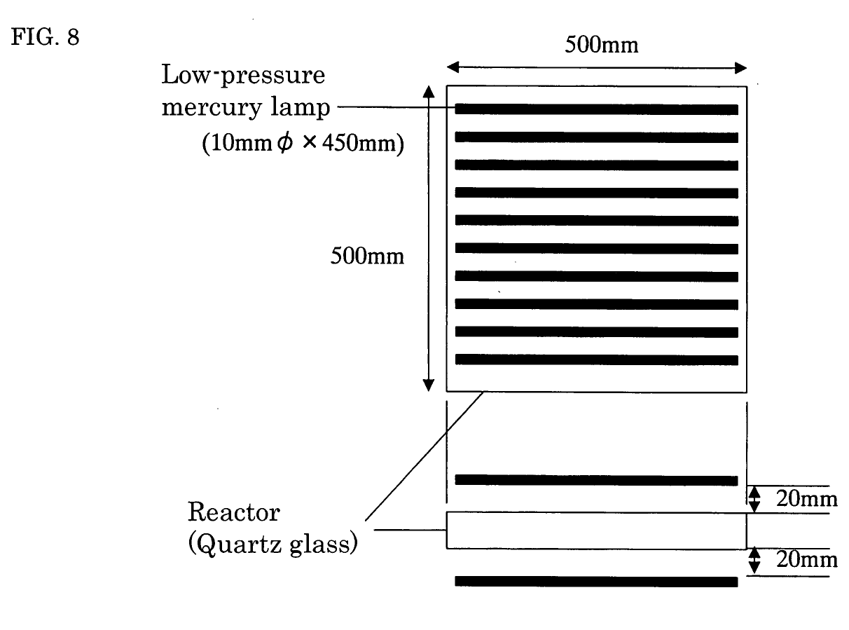

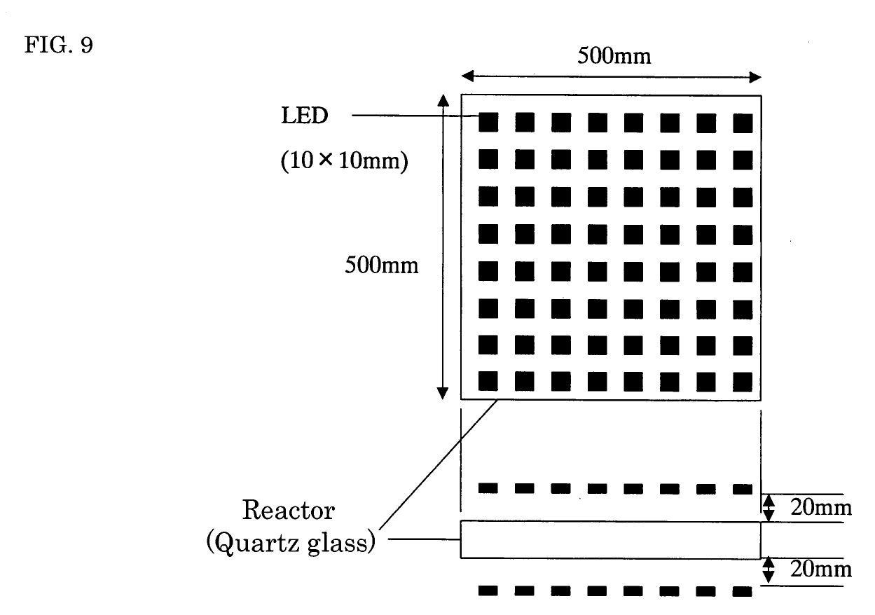

For the purpose of comparison, a device produced by laminating 50 layers of the photocatalytic woven fabric alone was set in the same container, and a light source, in which commercially available ultraviolet-emitting LEDs (luminescence wavelength 360 nm, output 50 mW) were arranged at a pitch of 35 mm (FIG. 9), and a light source, in which mercury lamps (luminescence wavelength 254 nm, output 100 mW) were arranged at a pitch of 35 mm (FIG. 8) were individually prepared and disposed outside the container. Irradiation was conducted from the outside of the container, and the lapse of time until complete decomposition was measured.

The results are shown in Table 3.

TABLE 3

| | | Amount | | | | | | | | | | |
|---|---|---|---|---|---|---|---|---|---|---|---|---|
| | | of second | | | | | | Lumines- | | | | Lapse of |
| First | Second | semicon- | | | | | Fluophor | cence | | | Addi- | time until |
| semicon- | semicon- | ductor | | | | | particle | wave- | Luminous | | tion of | complete |
| ductor | ductor | material | Donor | Acceptor | Grinding | Insulating | diameter | length | efficacy | Photo- | India | decomposi- |
| material | material | (mol %) | type | type | time (hr) | layer | (nm) | (nm) | (lm/W) | catalyst | ink | tion (hr) |
| ZnS | None | 0 | Cl | Ag | 18 | Cyano- | 2.5 | 355 | 10.4 | Anatase | Yes | 34 |
| | | | | | | resin + | | | | | | |
| | | | | | | BaTiO₃ | | | | | | |
| ZnS | None | 0 | Cl | Ag | 18 | Cyano- | 2.5 | 355 | 10.4 | Anatase | None | 8 |
| | | | | | | resin + | | | | | | |
| | | | | | | BaTiO₃ | | | | | | |

| | | | | | |
|---|---|---|---|---|---|
| GaN based LED | 360 | 40 | Anatase | Yes | 100< |
| Low-pressure mercury lamp | 254 | 80 | Anatase | Yes | 100< |
| GaN based LED | 360 | 40 | Anatase | None | 18 |
| Low-pressure mercury lamp | 254 | 80 | Anatase | None | 11 |

For the product according to the present aspect of the invention, the decomposition time was shorter than that for the external light source system. In particular, the difference was increased when a high-turbidity liquid was treated. The reason for this is believed to be that the radiated light is absorbed by the source of turbidity. Even when the turbidity is low, the decomposition time was short for the product according to the present aspect of the invention. The reason for this is believed to be that when the photocatalytic woven fabrics are laminated, the light does not uniformly reach an inside woven fabric. On the other hand, for the product according to the present aspect of the invention, it is believed that all photocatalytic woven fabrics uniformly perform their function regardless of the number of lamination because the light source is located in the vicinity of the photocatalytic woven fabric.

### INDUSTRIAL APPLICABILITY

The product according to an aspect of the present invention is a fiber capable of emitting ultraviolet light by, for example, the application of an alternating current voltage. When the product according to an aspect of the present invention is disposed in a turbid fluid and is allowed to perform its function, a photocatalytic reaction can be effected efficiently without using an external ultraviolet light source, e.g., an ultraviolet lamp or an ultraviolet LED. In particular, even in the case where a turbid fluid significantly absorbs ultraviolet and the external light source cannot cope with, it becomes possible to effect a photocatalytic reaction efficiently.

A photocatalytic reactor including the product according to an aspect of the present invention can decompose organic materials and sterilize bacteria and the like and, therefore, be applied to various fields, for example, decomposition and removal of NO_{X}, SO_{X}, a CO gas, diesel particulates, pollen, dust, acarid, and the like which become pollutants in air, decomposition and removal of organic compounds contained in sewage, light sources for sterilizing general bacteria, viruses, and the like, decomposition of harmful gases generated in chemical plants, decomposition of smelling components, and sterilization light source in ultra pure water plants.

It is possible to combine with a ceramic filter, a photocatalytic sheet, a photocatalytic woven fabric, or the like. For example, when the product according to an aspect of the present invention is disposed in a cell of a ceramic honeycomb filter allowed to carry a photocatalyst in advance, both the separating function of a ceramic filter and the photocatalytic function can be provided. For example, there is also a method in which the product according to an aspect of the present invention is disposed by being kitted into a photocatalytic woven fabric. In this manner, it is also possible to apply to honeycomb components used for automobile exhaust gas treatments, air cleaner filters, sewage filtration filters, various water purifiers, sterilization of hot spring, and insecticides.

1. An EL fiber having a function of emitting ultraviolet light or visible light with a wavelength of 400 nm or less, the EL fiber characterized in that the cross-sectional structure of the fiber comprises an internal electrode located at the center in a radius direction, an internal insulating layer disposed around the internal electrode, a light-emitting layer, an external electrode, and a protective layer disposed on an outermost surface, and the light is emitted by application of an alternating current electric field between the electrodes.

2. The EL fiber according to claim 1, wherein an external insulating layer is disposed between the light-emitting layer and the external electrode.

3. An EL fiber having a function of emitting ultraviolet light or visible light with a wavelength of 550 nm or less, the EL fiber characterized in that the cross-sectional structure of the fiber comprises an internal electrode located at the center in a radius direction, an internal insulating layer disposed around the internal electrode, a light-emitting layer, an external electrode, a protective layer, and a particle layer or a thin film disposed on an outermost surface and formed from a material having a photocatalytic function, and the light is emitted by application of an alternating current electric field between the electrodes.

4. The EL fiber according to claim 3, wherein an external insulating layer is disposed between the light-emitting layer and the external electrode.

5. The EL fiber according to claim 3, wherein the protective layer itself is formed from a material having a photocatalytic function.

6. The EL fiber according to claim 3, wherein the material having a photocatalytic function is TiO₂ and/or TiO₂ doped with at least one type of element of N, S, Mn, Fe, Co, Zn, and Cu.

7. The EL fiber according to claim 1, wherein the light-emitting layer has a structure in which fluophor particles having a function of emitting visible light or ultraviolet light are dispersed in a matrix containing at least one type of a dielectric resin and a dielectric ceramic.

8. The EL fiber according to claim 1, wherein the fluophor constituting the light-emitting layer containing ZnS as a first primary component and, as second components, a first additional element constituting an acceptor level and a second additional element constituting a donor level in a semiconductor partly including or not including a group II-IV compound semiconductor.

9. The EL fiber according to claim 8, wherein the first additional element is at least one type of Cu, Ag, Au, Li, Na, N, As, P, and Sb, and the second additional element is at least one type of Cl, Al, I, F, and Br.

10. The EL fiber according to claim 8, wherein the first additional element is Ag.

11. The EL fiber according to claim 8, wherein the semiconductor of the second component contains at least one type of MgS, CaS, SrS, BeS, and BaS.

12. The EL fiber according to any one of claim 1, wherein the average particle diameter of the fluophor constituting the light-emitting layer is 10 nm or less.

13. A photocatalytic reactor comprising the EL fiber according to claim 1.

14. A photocatalytic reactor having a structure in which the EL fiber according to claim 1 and a photocatalytic fiber are combined in alternate position.

15. The EL fiber according to claim 3, wherein the light-emitting layer has a structure in which fluophor particles having a function of emitting visible light or ultraviolet light are dispersed in a matrix containing at least one type of a dielectric resin and a dielectric ceramic.

16. The EL fiber according to claim 3, wherein the fluophor constituting the light-emitting layer containing ZnS as a first primary component and, as second components, a first additional element constituting an acceptor level and a second additional element constituting a donor level in a semiconductor partly including or not including a group II-IV compound semiconductor.

17. The EL fiber according to claim 3, wherein the average particle diameter of the fluophor constituting the light-emitting layer is 10 nm or less.

18. A photocatalytic reactor comprising the EL fiber according to claim 3.

19. A photocatalytic reactor having a structure in which the EL fiber according to claim 3 and a photocatalytic fiber are combined in alternate position.
